(19) United States
(12) Patent Application Publication
(10) Pub. No.: US 2009/0137569 A1
(43) Pub. Date: May 28, 2009

(54) Administration of (3S)-N-hydroxy-4-(sulfonyl)-2,2-dimethyl-3-thiomorpholine carboxamide or (S)-N_hydroxy-4-[4_(but-2-ynyloxy)phenylsulfonyl]-2,2-dimethyl thimomorpholine-3-carboxamide for preventing and/or treating inflammatory skin pathologies/afflictions

(75) Inventors: Thibaud Biadatti, Opio (FR); Marlene Schuppli, La Colle Sur Loup (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, BIOT (FR)

(21) Appl. No.: 12/232,456

(22) Filed: Sep. 17, 2008

(30) Foreign Application Priority Data
Mar. 16, 2007 (FR) PCT/FR2007/050939

(51) Int. Cl. A61K 31/54; A61P 17/00

(52) U.S. Cl. 514/227.5

(57) ABSTRACT

Inflammatory skin pathologies/afflictions, e.g., eczema or psoriasis, are prevented and/or treated by administering to a subject in need of such treatment, a thus effective amount of at least one compound of following formula (I):

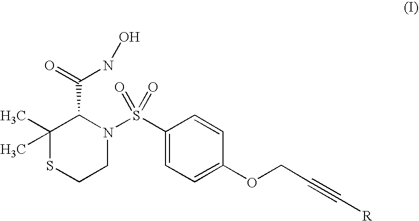

in which R is a —CH₃ radical or a CH₂OH radical, or of a pharmaceutically acceptable salt, solvate or hydrate thereof.

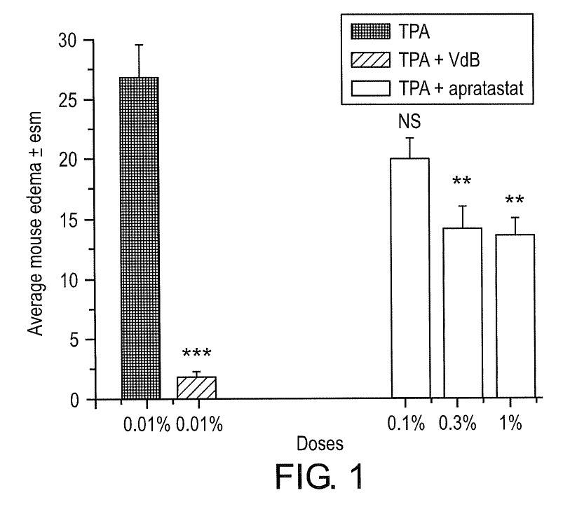

### CROSS-REFERENCE TO PRIORITY/PCT APPLICATIONS

This application claims priority under 35 U.S.C. § 119 of FR 06/02429, filed Mar. 20, 2006, and is a continuation/national phase of PCT/FR 2007/050939, filed Mar. 16, 2007 and designating the United States (published in the French language on Sep. 27, 2007 as WO 2007/107663 A2; the title and abstract were also published in English), each hereby expressly incorporated by reference in its entirety and each assigned to the assignee hereof.

### CROSS-REFERENCE TO COMPANION APPLICATION

Co-pending U.S. patent application Ser. No. ______ [Attorney Docket No. 1034227-000941], filed concurrently herewith, hereby expressly incorporated by reference and assigned to the assignee hereof.

### BACKGROUND OF THE INVENTION

1. Technical Field of the Invention

The present invention relates to the administration of a compound of the formula (I) or of one of its pharmaceutically acceptable salts, pharmaceutically acceptable solvates, or hydrates thereof, in a regime or regimen to prevent and/or treat inflammatory skin pathologies/afflictions, and particularly eczema or psoriasis.

2. Description of Background and/or Related and/or Prior Art

WO 00/44709 describes a family of hydroxamic acid arylsulfonamide derivatives, TNF-α converting enzyme inhibitors, which are useful in the treatment or prevention of arthritis, tumor metastases, tissue ulceration, abnormal healing of wounds, periodontal diseases, graft rejection, insulin resistance, bone diseases and AIDS.

### SUMMARY OF THE INVENTION

It is has now unexpectedly been found that certain of the compounds of this WO 00/44709 patent application are active in preventing and/or treating inflammatory skin pathologies/afflictions and particularly eczema or psoriasis.

### BRIEF DESCRIPTION OF THE DRAWING

The FIGURE of Drawing is a graph showing the modulation of TPA-induced mouse ear edema by VdB and by the compound (I) according to the invention.

### DETAILED DESCRIPTION OF BEST MODE AND SPECIFIC/PREFERRED EMBODIMENTS OF THE INVENTION

The present invention thus features administration of at least one compound selected from the compounds of following formula (I):

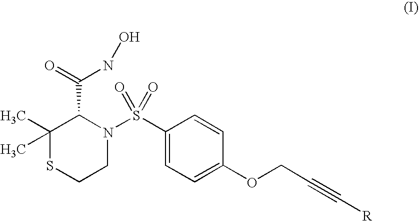

in which R is a —CH₃ radical or a CH₂OH radical,
their pharmaceutically acceptable salts, their pharmaceutically acceptable solvates or their hydrates thereof, in a regime or regimen to prevent and/or treat inflammatory skin pathologies/afflictions. These pathologies are in particular all forms of psoriasis, whether cutaneous, mucosal or ungual, psoriatic rheumatism, or also skin atopy, such as eczema, or respiratory atopy or also gingival hypertrophy.

The present invention more specifically features the administration of (3S)—N-hydroxy-4-({4-[(4-hydroxy-2-butynyl)oxy]phenyl}sulfonyl)-2,2-dimethyl-3-thiomorpholinecarboxamide (apratastat) or (S)—N-hydroxy-4-[4-(but-2-ynyloxy)phenylsulfonyl]-2,2-dimethylthiomorpholine-3-carboxamide or of one of its pharmaceutically acceptable salts, pharmaceutically acceptable solvates or hydrates thereof, in a regime or regimen to prevent and/or treat inflammatory skin pathologies/afflictions.

The salts of the compounds of formula (I) according to the invention comprise salts with organic or inorganic bases, for example alkali metal salts, such as lithium, sodium or potassium salts.

The term “hydrate of a compound of formula (I)” means a combination of this compound with one or more water molecules.

The term “solvate of a compound of formula (I)” means the combination resulting from the attachment of a solvent to the crystals of compound of formula (I) which are formed in the presence of this solvent.

For use as a medicament, the compounds of formula (I), their pharmaceutically acceptable salts, their pharmaceutically acceptable solvates or their hydrates should be formulated as a pharmaceutical composition, preferably a dermatological composition.

The present invention therefor also features pharmaceutical compositions, in particular dermatological compositions, comprising at least one compound selected from the compounds of formula (I):

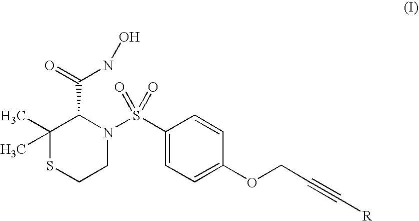

in which R is a —CH₃ radical or a CH₂OH radical,
their pharmaceutically acceptable salts, their pharmaceutically acceptable solvates or their hydrates, for the treatment and/or prevention of inflammatory skin pathologies and in particular all forms of psoriasis, whether cutaneous, mucosal or ungual, and even psoriatic rheumatism, or also skin atopy, such as eczema, or respiratory atopy or also gingival hypertrophy.

This invention more particularly features pharmaceutical compositions, in particular dermatological compositions, comprising at least one compound selected from the compounds of formula (I), their pharmaceutically acceptable salts, their pharmaceutically acceptable solvates or their hydrates, preferably for the treatment and/or prevention of eczema or psoriasis.

Such compositions are useful, and thus appropriate, for oral, topical, enteral, parenteral, ocular, sublingual, inhaled, subcutaneous, intramuscular, intravenous, transdermal, local or rectal administration. The compound of formula (I), optionally in the form of a salt, solvate and/or hydrate which is pharmaceutically acceptable, alone or in combination with another active principle, can be administered in a unit of administration form, as a mixture with conventional pharmaceutical carriers or excipients, to animals and human beings. Preferably, the pharmaceutical composition is packaged in a form suitable for oral or topical administration.

The compositions according to the invention comprise at least one compound of formula (I) or one of its pharmaceutically acceptable salts, pharmaceutically acceptable solvates or hydrates in amounts sufficient to obtain the desired prophylactic or therapeutic effect. The useful dosage varies according to the age, sex and weight of the patient.

The compound of formula (I) or one of its salts, solvates or hydrates will preferably be administered in a proportion of 0.01 to 100 mg/kg and per day, advantageously of 0.01 to 50 mg/kg and per day. It is also possible to administer such doses in 2 to 4 daily administrations. Although these dosages are examples of average situations, there may be specific cases where higher or lower dosages are appropriate; such dosages are also according to the invention.

The compositions according to the invention comprise a physiologically acceptable carrier or at least one pharmaceutically acceptable excipient selected according to the pharmaceutical form, in particular dermatological form, desired and the method of administration selected.

The term “physiologically acceptable carrier” and the term “pharmaceutically acceptable excipient” are understood to mean, respectively, a carrier and an excipient which are compatible with the skin, mucous membranes and superficial body growths.

For oral administration, the pharmaceutical or dermatological composition can be provided in the form of tablets, including sugar-coated tablets, hard gelatin capsules, pills, syrups, suspensions, solutions, powders, granules, emulsions, capsules or microspheres or nanospheres or lipid or polymeric vesicles which make possible controlled release.

Parenterally, the composition can be provided in the form of solutions or suspensions for infusion or for injection.

To obtain a solid composition for oral administration, the active principle can be mixed with at least one inert diluent, such as sucrose, lactose or starch. Generally, other additives, such as a lubricant, for example magnesium stearate, can be added. In the case of capsules, tablets or pills in particular, a buffer can be added. In the case of liquid oral compositions, an inert diluent, such as water, can be used.

Topically, the pharmaceutical composition according to the invention is more particularly useful for the treatment of the skin and mucous membranes and can be provided in the form of ointments, creams, milks, salves, powders, impregnated pads, syndets, solutions, gels, sprays, foams, suspensions, lotions, sticks, shampoos or washing bases. They can also be provided in the form of suspensions of microspheres or nanospheres or lipid or polymeric vesicles or of polymeric patches and of hydrogels which make possible controlled release. This topical composition can be provided in the anhydrous form, in the aqueous form or in the form of an emulsion.

The compound of formula (I) or one of its salts, solvates or hydrates, when it is administered orally, is administered in a proportion of 0.01 to 100 mg/kg and per day, advantageously of 0.01 to 50 mg/kg.

The compound of formula (I) or one of its salts, solvates or hydrates, when it is administered topically, is used at a concentration generally of from 0.001 to 10% by weight, preferably from 0.01 to 5% by weight, with respect to the total weight of the composition.

For the treatment of an inflammatory skin pathology and more particularly of eczema or psoriasis, at least one compound of formula (I) or one of its pharmaceutically acceptable salts or one of its pharmaceutically acceptable solvates or hydrates can be administered in combination with another active principle.

The pharmaceutical and dermatological compositions as described above can thus comprise inert additives or even pharmacodynamically active additives or combinations of these additives, and in particular:

wetting agent;

flavor enhancers;

preservatives, such as para-hydroxybenzoic acid esters;

stabilizing agents;

moisture regulators;

pH regulators;

osmotic pressure modifiers;

emulsifying agents;

UV-A and UV-B screening agents;

antioxidants, such as α-tocopherol, butylated hydroxyanisole or butylated hydroxytoluene, superoxide dismutase, ubiquinol or certain metal-chelating agents;

depigmenting agents, such as hydroquinone, azelaic acid, caffeic acid or kojic acid;

emollients;

moisturizing agents, such as glycerol, PEG 400, thiamorpholinone and its derivatives or urea;

antiseborrhoeic or anti-acne agents, such as S-carboxymethylcysteine, S-benzylcysteamine, their salts or their derivatives or benzyl peroxide;

antibiotics, such as erythromycin and its esters, neomycin, clindamycin and its esters, or tetracyclines;

antifungal agents, such as ketoconazole or 4,5-polymethylene-3-isothiazolidones;

agents which promote regrowth of the hair, such as minoxidil (2,4-diamino-6-piperidinopyrimidine 3-oxide) and its derivatives, diazoxide (7-chloro-3-methyl-1,2,4-benzothiadiazine 1,1-dioxide) and phenyloin (5,4-diphenylimidazolidine-2,4-dione);

nonsteroidal anti-inflammatory agents;

carotenoids and in particular β-carotene;

anti-psoriatic agents, such as anthralin and its derivatives;

eicosa-5,8,11,14-tetraynoic acid and eicosa-5,8,11-triynoic acid, their esters and amides;

retinoids, that is to say ligands of RAR or RXR receptors, natural or synthetic;

ligands of VDR receptors;

corticosteroids or oestrogens;

α-hydroxy acids and α-keto acids or their derivatives, such as lactic acid, malic acid, citric acid, glycolic acid, mandelic acid, tartaric acid, glyceric acid or ascorbic acid, and their salts, amides or esters, or β-hydroxy acids or their derivatives, such as salicylic acid and its salts, amides or esters;

blockers of ion channels, such as potassium channels;

or also, more particularly for pharmaceutical compositions, in combination with medicaments known for interfering with the immune system (for example cyclosporin, FK 506, glucocorticoids, monoclonal antibodies, soluble receptors, cytokines or growth factors, and the like).

Of course, one skilled in this art will take care to select the optional compound or compounds to be added to these compositions so that the desired effect on psoriasis or eczema is not, or not substantially, detrimentally affected by the envisaged addition.

The compounds of formula (I) are in particular synthesized as described in WO 00/44709.

The study of the properties of the compound of formula (I) has shown that the compound of formula (I) and its pharmaceutically acceptable salts, solvates or hydrates are not toxic and have an anti-inflammatory activity in the treatment of eczema and/or psoriasis which is expressed equally well topically as orally.

In order to further illustrate the present invention and the advantages thereof, the following specific examples are given, it being understood that same are intended only as illustrative and in nowise limitative. In said examples to follow, all parts and percentages are given by weight, unless otherwise indicated.

### Example 1

### Compositions

A—Oral Administration:

0.2 g tablet:

| | |
|---|---|
| Compound of formula (I) | 0.001 g |
| Starch | 0.114 g |
| Dicalcium phosphate | 0.020 g |
| Silica | 0.020 g |
| Lactose | 0.030 g |
| Talc | 0.010 g |
| Magnesium stearate | 0.005 g |

B—Topical Administration:

(a) Ointment:

| | |
|---|---|
| Compound of formula (I) | 0.30 g |
| White petrolatum, pharmaceutical grade | q.s. for 100 g |

(b) Lotion:

| | |
|---|---|
| Compound of formula (I) | 0.10 g |
| Polyethylene glycol (PEG 400) | 69.90 g |
| 95% Ethanol | 30.00 g |

### Example 2

### Evaluation of the Anti-Inflammatory Activity of Apratastat after a Single Topical Application in the TPA-Induced Mouse Ear Edema Test on Balb/c Mice (TPA=Tetradecanoylphorbol Acetate)

Treatment:

Edema is induced by single application of 20 μl of TPA dissolved in acetone at 0.01%.

The test compound is diluted in a TPA solution and applied at a concentration of 0.1%, 0.3% to 1%.

A positive control, betamethasone valerate (VdB), is also tested.

The thickness of the mouse ear is measured at T+6 h.

Results:

The results are presented in the FIGURE of Drawing.

After a single topical application of the positive control betamethasone valerate (0.01%), diluted in the TPA solution, a reduction in the edema of the ear of 93% (***) is observed.

Apratastat has a dose-dependent anti-inflammatory effect and reduces the edema of the ear induced by TPA by 25% (NS) (at 0.1%), 47% (**) (at 0.3%) and 49% (**) (at 1%).

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for preventing and/or treating an inflammatory skin pathology/affliction, comprising administering to a subject in need of such treatment, a thus effective amount of at least one compound selected from the group consisting of the compounds of formula (I): 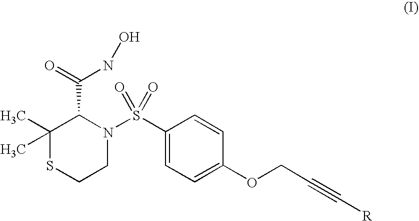
in which R is a —CH₃ radical or a CH₂OH radical, or a pharmaceutically acceptable salt, solvate or hydrate thereof, formulated into a physiologically/pharmaceutically acceptable carrier/excipient therefor.

2. The regime or regimen as defined by claim 1, said at least one compound of formula (I) comprising (3S)—N-hydroxy-4-({4-[(4-hydroxy-2-butynyl)oxy]phenyl}sulfonyl)-2,2-dimethyl-3-thiomorpholinecarboxamide.

3. The regime or regimen as defined by claim 1, said at least one compound of formula (I) comprising (S)—N-hydroxy-4-[4-(but-2-ynyloxy)phenylsulfonyl]-2,2-dimethylthiomorpholine-3-carboxamide.

4. The regime or regimen as defined by claim 1, comprising the treatment of eczema or psoriasis.

5. The regime or regimen as defined by claim 1, said at least one compound being formulated for oral administration.

6. The regime or regimen as defined by claim 1, said at least one compound being formulated for topical administration.

7. The regime or regimen as defined by claim 1, comprising the treatment of cutaneous, mucosal or ungual psoriasis, or psoriatic rheumatism.

8. The regime or regimen as defined by claim 1, comprising the treatment of skin or respiratory atopy, or gingival hypertrophy.
